SEQUENCE LISTING (12 sequences)

SEQ ID NO: 1 - DNA, 21 bp, artificial
  sense siRNA
gcaaccaaguuucuugaautt

SEQ ID NO: 2 - DNA, 21 bp, artificial
  antisense siRNA
auucaagaaacuugguugctt

SEQ ID NO: 3 - DNA, 21 bp, artificial
  sense siRNA
ccaaccaagugagaucaautt

SEQ ID NO: 4 - DNA, 21 bp, artificial
  antisense siRNA
auugaucucacuugguuggtt

SEQ ID NO: 5 - DNA, 21 bp, artificial
  sense siRNA
gccuuugguaagcuucaaatt

SEQ ID NO: 6 - DNA, 21 bp, artificial
  antisense siRNA
uuugaagcuuaccaaaggctt

SEQ ID NO: 7 - DNA, 21 bp, artificial
  sense siRNA
ggaucuuauuucuucggagtt

SEQ ID NO: 8 - DNA, 21 bp, artificial
  antisense siRNA
ttccuagaauaaagaagccuc

SEQ ID NO: 9 - DNA, 19 bp, artificial
  siRNA target sequence
gcaaccaagtttcttgaat

SEQ ID NO: 10 - DNA, 20 bp, artificial
  siRNA target sequence
accaaccaagtgagatcaat

SEQ ID NO: 11 - DNA, 19 bp, artificial
  siRNA target sequence
gcctttggtaagcttcaaa

SEQ ID NO: 12 - DNA, 730 bp, Bovine
atggaaaaatttgcacctgagtttcatggagaagatgccaatacaaaagcaaccaagtttcttgaatccctaaaagggaaatttacttcttctaaggattctaggaaaaaagatagtataatatcagttaattccgtagacatagaattacctaaagagagtcctataacatctaccaatcaaaatatcaaccaaccaagtgagatcaatgacactattgctacaaatcaagttcatataagaaagcctttggtaagcttcaaagaagaactgccatcaagtgaaaacccctttacaaggctgtataaggaaactatagaaacatttgacaataatgaagaagaatcaagctactcatatgatgagataaatgatcaaacaaatgataatataacagcaagactagataggatagatgaaaaattaagcgagataataggaatgctccatacattagttgtggctagtgcaggaccaacagctgctcgtgacggtataagagatgccatggtagggctccgagaagagatgattgagaaaataagatcagaagctttaatgaccaacgataggttagaagcaatggccaggcttagggatgaagaaagtgaaaagatgacaaaagatacatcagatgaagtaaaattaacccctacctcagagaagctgaacatggtattagaagatgaaagtagtgacaatgatctatcacttgaagatttctgaatag

(19) United States
(12) Patent Application Publication
(10) Pub. No.: US 2009/0053145 A1
(43) Pub. Date: Feb. 26, 2009

(54) ANTI-VIRAL COMPOSITIONS AND METHODS OF USE IN CATTLE

(75) Inventors: AMELIA R. WOOLUMS, WINDER, GA (US); RALPH A. TRIPP, WATKINSVILLE, GA (US)

Correspondence Address: MUETING, RAASCH & GEBHARDT, P.A., P.O. BOX 581336, MINNEAPOLIS, MN, 55458-1336, US

(73) Assignee: University of Georgia Research Foundation, Inc., Athens, GA (US)

(21) Appl. No.: 12/020,992

(22) Filed: Jan. 28, 2008

Related U.S. Application Data
(60) Provisional application No. 60/897,595, filed on Jan. 26, 2007.
(60) Provisional application No. 60/986,681, filed on Nov. 9, 2007.

(51) Int. Cl. A61K 9/12; C07H 21/02; C12N 15/63; A61K 31/7088

(52) U.S. Cl. 424/45; 536/23.1; 435/320.1; 435/325; 514/44

(57) ABSTRACT

Aspects of the present disclosure relate to therapeutic RNAi compositions and methods of use thereof for treating and/or preventing disease, disorder, and/or conditions—associated with bovine respiratory syncytial virus (BRSV) infections in cattle.

### RELATED APPLICATIONS

This application claims the benefit of priority of U.S. Provisional Patent Application Ser. Nos. 60/897, 595, filed Jan. 26, 2007 and 60/986,681, filed Nov. 9, 2007, the content of each of which is incorporated by reference in its entirety.

### FIELD

Aspects of the instant disclosure relate generally to therapeutic methods and compositions associated with viral gene modulation in cattle. In certain aspects, the instant disclosure is directed to methods and RNA interference (RNAi) compositions for the treatment of bovine respiratory syncytial virus (BRSV) and/or the Paramyxoviridae—associated disease, disorder, and/or conditions in cattle.

### BACKGROUND

The following includes information that may be useful in understanding the present invention. It is not an admission that any of the information provided herein is prior art, or relevant, to the presently described or claimed inventions, or that any publication or document that is specifically or implicitly referenced is prior art.

RNA interference (RNAi) is an evolutionarily conserved, gene-silencing mechanism wherein small double-stranded RNA molecules, or small interfering RNA (siRNA), targets cognate RNA for destruction with exquisite potency and selectivity causing post-transcriptional gene silencing. The RNAi machinery, which is expressed in all eukaryotic cells, has been shown to regulate the expression of key genes involved in cell differentiation in plants and animals. Given the ability of RNAi to silence genes, numerous RNAi based therapeutic strategies are being developed, particularly for RNA viruses such as HIV, influenza and respiratory syncytial virus. Development of synthetic siRNA drugs is particularly useful in situations in which long-term silencing is not required or undesirable, e.g. treating acute viral infections.

Homology-dependent gene silencing was discovered in transgenic plants in the form of co-suppression between introduced transgenes or between a transgene and its homologous endogenous gene. Both RNA silencing and RNAi are generic terms to describe gene silencing mechanisms guided by siRNAs and microRNAs (miRNA). The central feature of RNA silencing is the production of siRNAs by the endoribonuclease, Dicer. siRNAs are asymmetrically assembled into effector complexes called RNA-induced silencing complexes (RISC). siRNAs control the specificity of RNA silencing by recruiting the effector complex to a cognate single-stranded RNA target, leading to either slicing or translational arrest of nascent RNA synthesis.

Synthetic siRNAs delivered to a cell are incorporated into RISC complexes. Within the RISC complex, the two strands of the siRNA become separated, so that they can target complementary sequences in mRNAs. After pairing with an siRNA strand, the targeted mRNA is precisely cleaved and undergoes degradation thereby interrupting the synthesis of the targeted protein. The RISC complex is naturally stable within the cell, and once formed, will continue to seek and destroy the targeted mRNA molecules, resulting in sustained suppression of specific protein transcript synthesis. The antisense strand of siRNA directs endonuclease activity of RISC to the cognate mRNA target resulting in mRNA cleavage (Dykxhoorn et al., Nat. Rev. Mol. Cell Biol. 2003, 4: 457-67).

Viral pathogens of cattle are a serious cause of animal morbidity and financial loss worldwide, and agents such as foot and mouth disease virus (FMDV), rinderpest, and Rift Valley Fever virus are recognized to have potentially serious impact as agents of agroterrorism. Additionally, domestic viral disease of cattle cause significant impact in terms of morbidity, mortality, and decreased production. A major cause of financial loss to cattle producers is the bovine respiratory disease complex (BRDC). The BRDC is caused by primary viral infection followed by secondary bacterial infection, and exacerbated by management-related stressors. In the United States, the BRDC is the leading cause of morbidity and mortality in beef cattle, with cost to producers estimated as high as $3 billion annually. One of the major viral pathogens contributing to the BRDC is bovine respiratory syncytial virus (BRSV), which is a member of the Paramyxoviridae family. BRSV infection can cause significant and sometimes fatal disease alone as well as in conjunction with other pathogens. The agricultural and economic impact of BRSV is substantial, thus development of safe and effective vaccines and therapeutics remains a high priority. In spite of the importance of viral infections on a global and national scale, specific antiviral therapies are not currently available for use in cattle. Antiviral drugs used in human beings are precluded due to expense, toxicity, and concerns regarding development of resistance by off-target exposure of human pathogens.

### SUMMARY OF THE INVENTION

The inventions described and claimed herein have many aspects and attributes including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

Accordingly, aspects of the present disclosure include methods and compositions for treating, inhibiting and/or preventing disease, disorder, and/or conditions associated with bovine respiratory syncytial virus (BRSV) or other members of the Paramyxoviridae virus family in cattle. In certain aspects, methods and compositions for inhibiting or preventing BRSV infection and/or replication are provided. The inhibition can be achieved by administering a therapeutically effective amount of an RNAi polynucleotide molecule to a subject. The methods and compositions can include RNAi polynucleotides and/or nucleic acid molecules that bind to cognate BRSV RNA. The RNAi polynucleotide molecule can be double stranded.

### BRIEF DESCRIPTION OF THE FIGURES

FIGS. 1-10 illustrate Bovine respiratory syncytial virus (BSRV) challenge data to determine which method provided the most consistent signs of infection. Clinical challenge trial data shown in these figures indicate that clinical disease and lung pathology were induced with BRSV challenge (exposure) of calves. Two different methods of BRSV challenge (exposure) were used to determine which method would give the most consistent signs of disease. Two groups are described, representing the two different challenge methods. Group 1 had greater signs of disease and more lung pathology and, therefore, this challenge method is used for evaluating the RNAi treatment.

FIG. 1. Rectal temperatures for calves challenged with BRSV by DeVilbiss (Group 1) or Chromister (Group 2). Data are mean +/−SEM. Different letters indicate significant differences between groups (p<0.01 by repeated measures ANOVA).

FIG. 2. Respiratory rates for calves challenged with BRSV by DeVilbiss (Group 1) or Chromister (Group 2). Data are means +/−SEM. Different letters indicate significant differences (p<0.05 by repeated measures ANOVA).

FIG. 3. Average (+/−SEM) clinical scores for calves in Group 1 and 2. Different letters indicate significant differences (p<0.001 by repeated measures ANOVA).

FIG. 4. Mean (+/−SD) nasal BRSV shedding as identified by direct IFA score of cells collected from nasal swabs in calves challenged with BRSV by DeVilbiss (Group 1) or Chromister (Group 2).

FIG. 5. Real time reverse transcriptase PCR (RT-PCR) for BRSV N protein mRNA from nasal swabs from calves in Group 1 and Group 2. Values were determined by calculating ΔΔcT and expressing results as n-fold change from baseline (day 0). Values positive for N protein mRNA (n-fold change greater than 2) are highlighted.

FIG. 6. Nasal BRSV shedding as measured by real time reverse transcriptase PCR (RT-PCR) for BRSV N protein mRNA from RNA isolated from nasal swabs. Calves challenged with BRSV by DeVilbiss (Group 1) or Chromister (Group 2). Values were determined by calculating ΔΔcT and expressing results as n-fold change from baseline (day 0).

FIG. 7. Alternate version of graph of RT-PCR data, which makes smaller values more clear).

FIG. 8. Average (+/−SEM) lung weights (kg) for calves challenged with BRSV by DeVilbiss (Group 1) or Chromister (Group 2). Different letters indicate significant difference (p=0.03, Mann-Whitney test).

FIG. 9. Average (+/−SEM) gross lung pathology scores for calves challenged with BRSV by DeVilbiss (Group 1) or Chromister (Group 2). Different letters indicate significant difference, p=0.03 (Mann-Whitney test).

FIG. 10. Average (+/−SEM) histopathology scores for calves challenged with BRSV by DeVilbiss (Group 1) or Chromister (Group 2). Different letters indicate significant difference, p=0.02 (Mann-Whitney test).

FIG. 11 presents comparative data illustrating the efficacy of several small interfering RNA's (siRNAs). The results of real time RT-PCR for BRSV after transfection of BT cells with Ambion siRNA 1 or 3, or treatment with transfection reagent alone, followed by infection with BRSV SD B for 72 hours are illustrated. The data demonstrate that, relative to the control siRNA (1730), and relative to treatment with transfection reagent alone, treatment of cells with the Ambion 1 and 3 siRNAs (which are directed against BRSV mRNA) decreased levels of virus in cell culture in a dose-dependent fashion.

FIG. 12. BRSV plaque reduction by administration of siRNAs (100 nM). siRNA1, siRNA2, and siRNA3 were all directed to conserved regions of the BRSV P gene. NP 1496 was used as a negative control since it is specific for influenza virus.

### DETAILED DESCRIPTION

The instant disclosure relates to compounds, compositions, and methods useful for modulating gene expression using short interfering nucleic acid (siNA) molecules. The disclosure also relates to compounds, compositions, and methods of treatment useful for modulating the expression and/or activity of viral genes and or proteins in a subject by RNA interference (RNAi) using small nucleic acid molecules. In particular, the instant disclosure include small nucleic acid polynucleotide molecules (e.g. 19-27 nucleotides), short interfering nucleic acid (siNA), short interfering RNA (siRNA), double-stranded RNA (dsRNA), micro-RNA (miRNA), short hairpin RNA (shRNA), and antisense polynucleotide molecules and methods of use thereof to modulate the expression of genes associated with for viral replication or infection.

Definitions

As used herein, “subject” may include domestic, livestock, farm animals, and zoo, sports, or pet animals. The preferred subject is cattle (e.g. beef cattle and/or dairy cows, and calves), subjects may include members of the Bovidae family and/or members of the subfamily Bovinae. Exemplars can include members of Bos taurus, Bos indicus, or Bos prim igenius, and hybrid species thereof, including, for example, Bos primigenius taurus, Bos primigenius indicus and Bos primigenius primigenius.

As used herein, Bovine Respiratory Disease Complex (BRDC) can include any disease, disorder, and/or condition associated with, or mediated by, bovine respiratory syncytial virus (BRSV) and/or members of the pneumovirus genus and/or other members of the Paramyxoviridae family, and infections thereby. Exemplary BRSV-associated disease, disorder, and/or condition can include those characterized by fever; depression; decreased feed intake; increased respiratory rate; cough; nasal and lacrimal discharge; dyspnea (with or without open-mouthed breathing); subcutaneous emphysema; secondary bacterial pneumonia; a biphasic disease pattern; gross lesions, including a diffuse interstitial pneumonia with subpleural and interstitial emphysema along with interstitial edema; bronchopneumonia of bacterial origin; presence of syncytial cells in bronchiolar epithelium and/or lung parenchyma; intracytoplasmic inclusion bodies; proliferation and/or degeneration of bronchiolar epithelium; alveolar epithelialization; edema, and/or hyaline membrane formation.

As used herein, “preventing” means preventing in whole or in part, or ameliorating or controlling.

As used herein, the term “treating” or “treatment” or “alleviation” refers to both therapeutic treatment and prophylactic or preventative measures and “wherein the object is to prevent or slow down (lessen) the targeted pathologic condition or disorder. Those in need of treatment include those already with a viral infection as well as those prone to having an infection or those in which an infection is to be prevented.

As used herein, a “therapeutically effective amount” or “effective amount” in reference to the polynucleotides or compositions of the instant disclosure refers to the amount sufficient to induce a desired biological, pharmaceutical, or therapeutic result. The result can be alleviation of the signs, symptoms, or causes of a disease or disorder or condition, or any other desired alteration of a biological system. In certain embodiments, the result will involve preventing, retarding, or reducing the incidence or severity of and/or decreasing viral infection and/or viral replication in whole or in part. Generally, alleviation or treatment of a disease or disorder involves the lessening of one or more symptoms or medical problems associated with the disease or disorder.

As used herein, the phrase “duplex region” refers to the region in two complementary or substantially complementary polynucleotides that form base pairs with one another, either by Watson-Crick base pairing or any other manner that allows for a duplex between polynucleotide strands that are complementary or substantially complementary. For example, a polynucleotide strand having 25 nucleotide units can base pair with another polynucleotide of 25 nucleotide units, yet only 23 bases on each strand are complementary or substantially complementary, such that the “duplex region” consists of 23 base pairs. The remaining base pairs may, for example, exist as 5′ and 3′ overhangs. Further, within the duplex region, 100% complementarity is not required; substantial complementarity is allowable within a duplex region. Substantial complementarity refers to complementarity between the strands such that they are capable of annealing under biological conditions. Techniques to empirically determine if two strands are capable of annealing under biological conditions are well know in the art. Alternatively, two strands can be synthesized and added together under biological conditions to determine if they anneal to one another.

As used herein, an siRNA having a sequence “sufficiently complementary” to a target mRNA sequence means that the siRNA has a sequence sufficient to trigger the destruction of the target mRNA by the RNAi machinery (e.g., the RISC complex) or process. The siRNA molecule can be designed such that every residue of the antisense strand is complementary to a residue in the target molecule. Alternatively, substitutions can be made within the molecule to increase stability and/or enhance processing activity of said molecule. Substitutions can be made within the strand or can be made to residues at the ends of the strand.

As used herein, “operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

Anti-Viral Polynucleotides and Agents

Exemplary anti-viral agents include agents that decrease or inhibit expression or function of viral genes/proteins associated with replication and/or infection. Anti-viral agents include anti-viral polynucleotides, such as, for example, siRNA, shRNA, and miRNA polynucleotides and/or other polynucleotides having RNAi, antisense, ribozyme, or inhibitory functionalities). In addition, anti-viral agents can also include antibodies and binding fragments thereof, and peptides and polypeptides, including peptidomimetics and peptide analogs that modulate and/or target viral gene/protein activity or function in a sequence-specific manner.

Synthesis of anti-viral polynucleotides such as siRNA, shRNA, miRNA, and ribozyme polynucleotides, as well as polynucleotides having modified and mixed backbones, is well-known to those of skill in the art. See e.g. Stein C. A. and Krieg A. M. (eds), Applied SiRNA Oligonucleotide Technology, 1998 (Wiley-Liss). Methods of synthesizing sequence specific antibodies and binding fragments, as well as peptides and polypeptides, including peptidomimetics and peptide analogs, are known to those of skill in the art. See e.g. Lihu Yang et al., Proc. Natl. Acad. Sci. U.S.A., 1; 95(18): 10836-10841 (Sept 1 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

In one aspect, the silencing or the downregulation of viral protein expression may be based generally upon the RNAi approach using RNAi polynucleotides (such as siRNA, miRNA, shRNA polynucleotides). These polynucleotides target the viral gene(s)/protein (s) to be silenced and/or downregulated. In certain embodiments, modulation of the viral protein expression comprises the silencing and/or downregulation of the target viral gene and may be based generally upon the siRNA approach using siRNA polynucleotides.

In certain embodiments, the RNAi polynucleotides can inhibit transcription and/or translation of a viral protein. Preferably the polynucleotide is a specific inhibitor of transcription and/or translation from the viral gene, and does not inhibit transcription and/or translation from other genes or mRNAs. The product may bind to the viral gene either (i) 5′ to the coding sequence, and/or (ii) to the coding sequence, and/or (iii) 3′ to the coding sequence.

In certain embodiments, the RNAi polynucleotide, such as siRNA polynucleotide, is directed to a viral protein mRNA. Such a polynucleotide may be capable of hybridizing to the viral mRNA and may thus inhibit the expression of viral by interfering with one or more aspects of viral mRNA metabolism including transcription, mRNA processing, mRNA transport from the nucleus, translation or mRNA degradation. The siRNA polynucleotide typically hybridizes to the viral mRNA to form a duplex which can cause direct inhibition of translation and/or destabilization of the mRNA.

In certain embodiments, the RNAi polynucleotide, such as siRNA polynucleotide, may hybridize to all or part of the target viral RNA. Typically the siRNA polynucleotide hybridizes to the ribosome binding region or the coding region of the viral mRNA. The polynucleotide may be complementary to all of or a region of the viral mRNA. For example, the polynucleotide may be the exact complement of all or a part of viral mRNA. However, absolute complementarity is not required and polynucleotides which have sufficient complementarity to form a duplex having a melting temperature of greater than about 20° C., 30° C. or 40° C. under physiological conditions are particularly suitable for use in the present invention.

Thus the polynucleotide is typically a homologue of a sequence complementary to the mRNA. The polynucleotide may be a polynucleotide which hybridizes to the viral mRNA under conditions of medium to high stringency such as 0.03M sodium chloride and 0.03M sodium citrate at from about 50° C. to about 60° C.

In certain embodiments, suitable polynucleotides are typically from about 19 to 30 nucleotides in length. In other embodiments, a polynucleotide may be from about 19 to about 27 nucleotides in length, or alternatively from about 19 to about 25 nucleotides in length or from about 19 to about 22 nucleotides in length.

The viral protein or proteins targeted by the polynucleotide will be dependent upon the site at which silencing/downregulation is to be effected.

It is also contemplated that polynucleotides targeted to separate viral proteins be used in combination (for example 1, 2, 3, 4 or more different viral proteins may be targeted).

Alternatively, the polynucleotides may be part of compositions that may comprise polynucleotides to more than one viral protein.

Individual siRNA polynucleotides may be specific to a particular viral gene, or may target 1, 2, 3 or more different viral genes according to varying degrees of sequence homology and conserved sequences.

In general, short interfering RNAs (siRNAs) typically comprise 19-27 nucleotide complementary double stranded RNA molecules with 2 nucleotide overhangs on the 3-prime ends of the molecules (de Fougerolles, A., H.-P. Vornlocher, J. Maraganore, and J. Lieberman. 2007. Interfering with disease: a progress report on siRNA-based therapeutics. Nat Rev Drug Discov 6:443-453; Amarzguioui, M., J. J. Rossi, and D. Kim. 2005. Approaches for chemically synthesized siRNA and vector-mediated RNAi. FEBS Letters 579:5974-5981). These compositions can be produced using a variety of tools including, but not limited to chemical synthesis (de Fougerolles, A., H.-P. Vornlocher, J. Maraganore, and J. Lieberman. 2007. Interfering with disease: a progress report on siRNA-based therapeutics. Nat Rev Drug Discov 6:443-453; Ronald, M. 2002. Small Interfering RNAs and Their Chemical Synthesis. Angewandte Chemie International Edition 41:2265-2269; Manoharan, M. 2004. RNA interference and chemically modified small interfering RNAs. Current Opinion in Chemical Biology 8:570-579; Davis, R. H. 1995. Large-scale oligoribonucleotide production. Current Opinion in Biotechnology 6:213-217), shRNA and miRNA expression vectors followed by processing in vivo (Amarzguioui, M., J. J. Rossi, and D. Kim. 2005. Approaches for chemically synthesized siRNA and vector-mediated RNAi. FEBS Letters 579:5974-5981), and in vitro transcription (Sohail, M., G. Doran, J. Riedemann, V. Macaulay, and E. M. Southern. 2003. A simple and cost-effective method for producing small interfering RNAs with high efficacy. Nucl. Acids Res. 31 :e38-).

The polynucleotides for use in the invention may suitably be unmodified phosphodiester oligomers. Such polynucleotides may vary in length.

In certain embodiments, the exemplary RNAi polynucleotides may also be chemically modified to improve stability, delivery, and efficacy (Li, C. X., A. Parker, E. Menocal, S. Xiang, L. Borodyansky, and J. H. Fruehauf 2006. Delivery of RNA interference. Cell Cycle 5:2103-2109). Methods of preparing modified backbone and mixed backbone oligonucleotides are known in the art. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy-or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. In certain embodiments, the chemical modifications may include but are not limited to 2′-Oallyl, and 2′-deoxyfluorouridine modifications, phosphothioates, 2′deoxyfluoridine (2′-F) modification, and locked nucleic acid residues (Li, C. X., A. Parker, E. Menocal, S. Xiang, L. Borodyansky, and J. H. Fruehauf. 2006. Delivery of RNA interference. Cell Cycle 5:2103-2109; de Fougerolles, A., H.-P. Vomlocher, J. Maraganore, and J. Lieberman. 2007. Interfering with disease: a progress report on siRNA-based therapeutics. Nat Rev Drug Discov 6:443-453; Manoharan, M. 2004. RNA interference and chemically modified small interfering RNAs. Current Opinion in Chemical Biology 8:570-579). In certain embodiments, silyl ether protection of the polynucleotide can also be used.

The precise sequence of the siRNA polynucleotide used in the invention will depend upon the target viral protein. In one embodiment, suitable viral siRNA polynucleotides can include polynucleotides selected from the following sequences set forth in Table 1.

TABLE 1

Exemplary siRNAs Targeting BRSV (Accession
# NC00189.1) and/or BRSV P gene (NC_001989
REGION: 2334 . . . 3202)

| | | SEQUENCE | Target |
|---|---|---|---|
| siRNAs | siRNA Sequence | ID Nos. | Accession # |
| siRNA1 | 5′ GCAACCAAGUUUCUU | 1 | NC00189.1 |
| sense | GAAUtt-3′ | | NC 001989 |
| siRNA1 | 3′- ttCGUUGGUUCAAA | 2 | NC00189.1 |
| antisense | GAACUUA-5′ | | NC 001989 |
| siRNA2 | 5′- CCAACCAAGUGAGA | 3 | NC00189.1 |
| sense | UCAAUtt-3′ | | NC 001989 |
| siRNA2 | 3′- ttGGUUGGUUCACU | 4 | NC00189.1 |
| antisense | CUAGUUA-5′ | | NC 001989 |
| siRNA3 | 5′- GCCUUUGGUAAGCU | 5 | NC00189.1 |
| sense | UCAAAtt-3′ | | NC 001989 |
| siRNA3 | 3′-ttCGGAAACCAUUCG | 6 | NC00189.1 |
| antisense | AAGUUU-5′ | | NC 001989 |
| negative | 5′ GGAUCUUAUUUCUUC | 7 | |
| control | GGAGdTdT3′ | | |
| siRNA | | | |
| specific | | | |
| to influenza | | | |
| nucleoprotein | | | |
| NP-1496 | | | |
| (siNP): | | | |
| sense | | | |
| negative | 5′ dTdTCCUAGAAUAAA | 8 | |
| control | GAAGCCUC3′ | | |
| siRNA | | | |
| specific | | | |
| to influenza | | | |
| nucleoprotein | | | |
| NP-1496 | | | |
| (siNP): | | | |
| antisense | | | |

The precise length of the the siRNA can be varied according to hybridization and therapeutic efficacy. For example, in certain embodiments, siRNA 1 (target seq: gcaaccaagttt cttgaat) can additionally include at least 1, 2, 3, 4, or 5nt upstream (e.g. caaaa) or at least, 1, 2, 3, 4, or 5 nts downstream (e.g. cccta). For siRNA 2 (target seq:target seq:accaaccaag tgagatcaat), the siRNA can can additionally include at least 1, 2, 3, 4, or 5nt upstream (e.g. tatca) or at least, 1, 2, 3, 4, or 5 nts downstream (e.g. gacac). For siRNA 3 (target seq: gcctt tggtaagctt caaa), the siRNA can additionally include at least 1, 2, 3, 4, or 5nt upstream (e.g. agaaa) and/or at least, 1, 2, 3, 4, or 5 nts downstream (e.g. gaaga).

Exemplary sequence of the target Bovine Respiratory Syncythial Virus BRSV is described in Accession Number (NC 001989): (siRNAs target regions shown in bold)

5′atggaaaaat ttgcacctga gtttcatgga gaagatgcca
atacaaaagc aaccaagttt cttgaatccc taaaagggaa
atttacttct tctaaggatt ctaggaaaaa agatagtata
atatcagtta attccgtaga catagaatta cctaaagaga
gtcctataac atctaccaat caaaatatca accaaccaag
tgagatcaat gacactattg ctacaaatca agttcatata
agaaagcctt tggtaagctt caaagaagaa ctgccatcaa
gtgaaaaccc ctttacaagg ctgtataagg aaactataga
aacatttgac aataatgaag aagaatcaag ctactcatat
gatgagataa atgatcaaac aaatgataat ataacagcaa
gactagatag gatagatgaa aaattaagcg agataatagg
aatgctccat acattagttg tggctagtgc aggaccaaca
gctgctcgtg acggtataag agatgccatg gtagggctcc
gagaagagat gattgagaaa ataagatcag aagctttaat
gaccaacgat aggttagaag caatggccag gcttagggat
gaagaaagtg aaaagatgac aaaagataca tcagatgaag
taaaattaac ccctacctca gagaagctga acatggtatt
agaagatgaa agtagtgaca atgatctatc acttgaagat
ttctgaatag ′3

Anti-viral polynucleotides directed to viral proteins can be selected in terms of their nucleotide sequence by any convenient, and conventional, approach. For example, the BLAST search engine at National Center for Biotechnology Information (NCBI). Once selected, the RNAi polynucleotides can be synthesized using a DNA synthesizer.

In one embodiment of the invention, interfering RNA (e.g., siRNA) has a sense strand and an antisense strand, and the sense and antisense strands comprise a region of at least near-perfect contiguous complementarity of at least 19 nucleotides.

In a further embodiment, interfering RNA (e.g., siRNA) has a sense strand and an antisense strand, and the antisense strand comprises a region of at least near-perfect contiguous complementarity of at least 19 nucleotides to a target sequence, and the sense strand comprises a region of at least near-perfect contiguous identity of at least 19 nucleotides with a target sequence of target mRNA, respectively.

The length of each strand of the interfering RNA can comprise 19 to 27 nucleotides, and may comprise a length of 19, 20, 21, 22, 23, 24, 25, 26, or 27 nucleotides.

Interfering RNA target sequences (e.g., siRNA target sequences) within a target mRNA sequence can be selected using available design tools. Interfering RNAs corresponding to a target sequence can then be tested by transfection of cells expressing the target mRNA followed by assessment of knockdown using methods well known in the art.

As used herein, the strands of a double-stranded interfering RNA (e.g., an siRNA) may be connected to form a hairpin or stem-loop structure (e.g., an shRNA).

Nucleotides at the 3′ end of the sense strand may be deoxynucleotides for enhanced processing. Design of dicer-substrate 27-mer duplexes from 19-21 nucleotide target sequences, such as provided herein, is further discussed by the Integrated DNA Technologies (IDT) website and by Kim, D.-H. et al., (February, 2005) Nature Biotechnology 23:2; 222-226.

In certain embodiments, when interfering RNAs are produced by chemical synthesis, phosphorylation at the 5′ position of the nucleotide at the 5′ end of one or both strands (when present) can be added to enhance siRNA efficacy and specificity of the bound RISC complex.

One of skill in the art is able to use the target/sequence information provided in Tables 1 to design interfering RNAs having a length shorter or longer than the sequences provided in the table.

The target sequence in the mRNAs corresponding to target viral genes may be in the 5′ or 3′ untranslated regions of the mRNA as well as in the coding region of the mRNA.

One or both of the strands of double-stranded interfering RNA may have a 3′ overhang of from 1 to 6 nucleotides, which may be ribonucleotides or deoxyribonucleotides or a mixture thereof. The nucleotides of the overhang are not base-paired. In one embodiment of the invention, the interfering RNA comprises a 3′ overhang of TT or UU. In another embodiment of the invention, the interfering RNA comprises at least one blunt end. The termini usually have a 5′ phosphate group or a 3′ hydroxyl group. In other embodiments, the antisense strand has a 5′ phosphate group, and the sense strand has a 5′ hydroxyl group. In still other embodiments, the termini are further modified by covalent addition of other molecules or functional groups.

The sense and antisense strands of the double-stranded siRNA may be in a duplex formation of two single strands as described above or may be a single molecule where the regions of complementarity are base-paired and are covalently linked by a hairpin loop so as to form a single strand. It is believed that the hairpin is cleaved intracellularly by a protein termed dicer to form an interfering RNA of two individual base-paired RNA molecules.

Interfering RNAs may differ from naturally-occurring RNA by the addition, deletion, substitution or modification of one or more nucleotides. Non-nucleotide material may be bound to the interfering RNA, either at the 5′ end, the 3′ end, or internally. Such modifications are commonly designed to increase the nuclease resistance of the interfering RNAs, to improve cellular uptake, to enhance cellular targeting, to assist in tracing the interfering RNA, to further improve stability, or to reduce the potential for activation of the interferon pathway. For example, interfering RNAs may comprise a purine nucleotide at the ends of overhangs. Conjugation of cholesterol to the 3′ end of the sense strand of an siRNA molecule by means of a pyrrolidine linker, for example, also provides stability to an siRNA.

Further modifications include a 3′ terminal biotin molecule, a peptide known to have cell-penetrating properties, a nanoparticle, a peptidomimetic, a fluorescent dye, or a dendrimer, for example.

Nucleotides may be modified on their base portion, on their sugar portion, or on the phosphate portion of the molecule and function in embodiments of the present invention.

Modifications include substitutions with alkyl, alkoxy, amino, deaza, halo, hydroxyl, thiol groups, or a combination thereof, for example. Nucleotides may be substituted with analogs with greater stability such as replacing a ribonucleotide with a deoxyribonucleotide, or having sugar modifications such as 2′ OH groups replaced by 2′ amino groups, 2′ 0-methyl groups, 2′ methoxyethyl groups, or a 2′-O, 4′-C methylene bridge, for example. Examples of a purine or pyrimidine analog of nucleotides include a xanthine, a hypoxanthine, an azapurine, a methylthioadenine, 7-deaza-adenosine and O- and N-modified nucleotides. The phosphate group of the nucleotide may be modified by substituting one or more of the oxygens of the phosphate group with nitrogen or with sulfur (phosphorothioates). Modifications are useful, for example, to enhance function, to improve stability or permeability, or to direct localization or targeting.

There may be a region or regions of the antisense interfering RNA strand that is (are) not complementary to a portion of the target viral genes. Non-complementary regions may be at the 3′, 5′ or both ends of a complementary region or between two complementary regions.

Interfering RNAs may be generated exogenously by chemical synthesis, by in vitro transcription, or by cleavage of longer double-stranded RNA with dicer or another appropriate nuclease with similar activity. Chemically synthesized interfering RNAs, produced from protected ribonucleoside phosphoramidites using a conventional DNA/RNA synthesizer, may be obtained from commercial suppliers such as Ambion Inc. (Austin, Tex.), Invitrogen (Carlsbad, Calif.), or Dharmacon (Lafayette, Colo.). Interfering RNAs are purified by extraction with a solvent or resin, precipitation, electrophoresis, chromatography, or a combination thereof, for example. Alternatively, interfering RNA may be used with little if any purification to avoid losses due to sample processing.

Interfering RNAs can also be expressed endogenously from plasmid or viral expression vectors or from minimal expression cassettes, for example, PCR generated fragments comprising one or more promoters and an appropriate template or templates for the interfering RNA. Examples of commercially available plasmid-based expression vectors for shRNA include members of the pSilencer series (Ambion, Austin, Tex.) and pCpG-siRNA (InvivoGen, San Diego, Calif.). Viral vectors for expression of interfering RNA may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT®DEST (Invitrogen, Carlsbad, Calif.). Selection of viral vectors, methods for expressing the interfering RNA from the vector and methods of delivering the viral vector are within the ordinary skill of one in the art. Examples of kits for production of PCR-generated shRNA expression cassettes include Silencer Express (Ambion, Austin, Tex.) and siXpress (Mirus, Madison, Wis.). A first interfering RNA may be administered via in vivo expression from a first expression vector capable of expressing the first interfering RNA and a second interfering RNA may be administered via in vivo expression from a second expression vector capable of expressing the second interfering RNA, or both interfering RNAs may be administered via in vivo expression from a single expression vector capable of expressing both interfering RNAs.

Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

Hybridization under Physiological Conditions: In certain embodiments of the present invention, an antisense strand of an interfering RNA hybridizes with an mRNA in vivo as part of the RISC complex.

For example, high stringency conditions could occur at about 50% formamide at 37° C. to 42° C. Reduced stringency conditions could occur at about 35% to 25% formamide at 30° C. to 35° C. Examples of stringency conditions for hybridization are provided in Sambrook, J., 1989, Molecular Cloning: A Laboratory Manual, Cold Spring Harbor Laboratory Press, Cold Spring Harbor, N.Y. Further examples of stringent hybridization conditions include 400 mM NaCl, 40 mM PIPES pH 6.4, 1 mM EDTA, 50° C. or 70° C. for 12-16 hours followed by washing, or hybridization at 70° C. in 1×SSC or 50° C. in 1×SSC, 50% formamide followed by washing at 70° C. in 0.3×SSC, or hybridization at 70° C. in 4×SSC or 50° C. in 4×SSC, 50% formamide followed by washing at 67° C. in 1 ×SSC. The temperature for hybridization is about 5-10° C. less than the melting temperature (Tₘ) of the hybrid where Tₘ is determined for hybrids between 19 and 49 base pairs in length using the following calculation: Tₘ° C.=81.5+16.6(logio[Na+])+0.41 (% G+C)−(600/N) where N is the number of bases in the hybrid, and [Na+] is the concentration of sodium ions in the hybridization buffer.

Single-stranded interfering RNA: As cited above, interfering RNAs ultimately function as single strands. Single-stranded (ss) interfering RNA has been found to effect mRNA silencing, albeit less efficiently than double-stranded RNA. Therefore, embodiments of the present invention also provide for administration of a ss interfering RNA that hybridizes under physiological conditions to a portion of the target RNA.

SS interfering RNAs are synthesized chemically or by in vitro transcription or expressed endogenously from vectors or expression cassettes as for ds interfering RNAs. 5′ Phosphate groups may be added via a kinase, or a 5′ phosphate may be the result of nuclease cleavage of an RNA. Delivery is as for ds interfering RNAs. In one embodiment, ss interfering RNAs having protected ends and nuclease resistant modifications are administered for silencing. SS interfering RNAs may be dried for storage or dissolved in an aqueous solution. The solution may contain buffers or salts to inhibit annealing or for stabilization.

Hairpin interfering RNA: A hairpin interfering RNA is a single molecule (e.g., a single oligonucleotide chain) that comprises both the sense and antisense strands of an interfering RNA in a stem-loop or hairpin structure (e.g., an shRNA). For example, shRNAs can be expressed from DNA vectors in which the DNA oligonucleotides encoding a sense interfering RNA strand are linked to the DNA oligonucleotides encoding the reverse complementary antisense interfering RNA strand by a short spacer. If needed for the chosen expression vector, 3′ terminal T's and nucleotides forming restriction sites may be added. The resulting RNA transcript folds back onto itself to form a stem-loop structure.

Techniques for selecting target sequences for siRNAs are provided by Tuschl, T. et al., “The siRNA User Guide,” revised May 6, 2004, available on the Rockefeller University web site; by Technical Bulletin #506, “siRNA Design Guidelines,” Ambion Inc. at Ambion's web site; and by other web-based design tools at, for example, the Invitrogen, Dharmacon, Integrated DNA Technologies, Genscript, or Proligo web sites. Initial search parameters can include G/C contents between 35% and 55% and siRNA lengths between 19 and 27 nucleotides. The target sequence may be located in the coding region or in the 5′ or 3′ untranslated regions of the mRNAs.

Polynucleotide Homologues

Homology and homologues are discussed herein (for example, the polynucleotide may be a homologue of a complement to a sequence in viral mRNA). Such a polynucleotide typically has at least about 70% homology, preferably at least about 80%, at least about 90%, at least about 95%, at least about 97% or at least about 99% homology with the relevant sequence, for example over a region of at least about 15, at least about 20, at least about 25 contiguous nucleotides (of the homologous sequence).

Homology may be calculated based on any method in the art. For example the UWGCG Package provides the BESTFIT program which can be used to calculate homology (for example used on its default settings) (Devereux et al (1984) Nucleic Acids Research 12, p387-395). The PILEUP and BLAST algorithms can be used to calculate homology or line up sequences (typically on their default settings), for example as described in Altschul S. F. (1993) J Mol Evol 36: 290-300; Altschul, S, F et al (1990) J Mol Biol 215: 403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighborhood word score threshold (Altschul et al, supra). These initial neighborhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached.

The BLAST algorithm parameters W, T and X determine the sensitivity and speed of the alignment. The BLAST program uses as defaults a word length (W), the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to a second sequence is less than about 1, preferably less than about 0. 1, more preferably less than about 0.01, and most preferably less than about 0.001.

The homologous sequence typically differs from the relevant sequence by no more than about 1, 2, 3, 4, 5, 6, 7, 8, 9, 10, 15, or 20 more mutations (which may be substitutions, deletions or insertions). These mutations may be measured across any of the regions mentioned above in relation to calculating homology.

The homologous sequence typically hybridizes selectively to the original sequence at a level significantly above background. Selective hybridization is typically achieved using conditions of medium to high stringency (for example 0.03M sodium chloride and 0.03M sodium citrate at from about 50° C. to about 60° C.). However, such hybridization may be carried out under any suitable conditions known in the art (see Sambrook et al. (1989), Molecular Cloning: A Laboratory Manual). For example, if high stringency is required, suitable conditions include 0.2×SSC at 60° C. If lower stringency is required, suitable conditions include 2×SSC at 60° C.

Peptide and Polypeptide Anti-Viral Agents

As used herein, polypeptide and polypeptide anti-viral agents can include binding proteins, including peptides, peptidomimetics, antibodies, antibody fragments, and the like, are also suitable modulators of viral gene/protein functions. Exemplary peptide and polypeptide anti-viral agents can modulate the structure, function and/or activity of the viral proteins encoded by the sequences and sequence homologs thereof as described in Table 2.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (i.e., that portion of a molecule, generally referred to as an epitope) that makes contact with a particular antibody or other binding molecule. These binding proteins, including antibodies, antibody fragments, and so on, may be chimeric or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g. viral infection and/or replication protein or associated molecules).

Binding molecules will generally have a desired specificity, including but not limited to binding specificity, and desired affinity. Affinity, for example, may be a Ka of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins according to the present invention can be readily determined using conventional techniques, for example those described by Scatchard et al., 1949 Ann. N.Y. Acad. Sci. 51: 660.

General Methodology

While vaccines for BRSV exist, it is recognized that vaccination can occasionally enhance disease and that improved means of protection against BRSV are needed. In vitro studies and mouse studies have shown that the use of appropriate dsRNA siRNA can mediate this response and lead to a decrease in target protein expression, thus mediating inhibition of viral replication and virus-induced disease (Bitko & Bank, BMC Microbiol. 2001, 1:1:34-45; Bitko et al., Nat. Med. 2005, 11:50-55; Ge et al., Proc. Natl. Acad. Sci. USA 2003, 100:2718-2723; Jacque et al, Nature. 2002. 418: 435-438). More specifically, mice prophylactically treated with siRNA directed against viral RNA-dependent RNA polymerase, i.e. the RSV P protein that is necessary for transcription of the viral genome, had lower respiratory rates, less lung pathology, and less virus isolated from lungs post-mortem than mice treated with an irrelevant siRNA (Bitko et al., 2005).

An antiviral strategy feasible for use in cattle and other agricultural animals is the use of small interfering RNA's (siRNA). The feasibility of using siRNA to prevent BRSV infection in cattle is indicated by studies confirming the efficacy of siRNA to block replication of the closely related human respiratory syncytial virus (RSV) in vitro and in mice. siRNA is an attractive antiviral strategy for use in animals because manufacture of the required ˜20 basepair RNAs is relatively simple. Candidate siRNAs can be tested rapidly in vitro to predict their in vivo efficacy. Moreover, as the technology becomes widespread, the cost of production can be expected to decrease. Thus siRNA is a potentially valuable tool for rapid control of outbreaks of viral disease in animals. However, to date there are no published reports describing the use of siRNA to prevent viral infection in cattle.

Accordingly, aspects and embodiments of the present disclosure is directed to methods and compositions that contain double stranded RNA (“dsRNA”), and methods of use thereof that are capable of reducing the expression of target genes in eukaryotic cells. One of the strands of the dsRNA contains a region of nucleotide sequence that has a length that ranges from about 19 to about 27 nucleotides that can direct the destruction of the RNA transcribed from the target gene.

Exemplary General Methodology for RNAi Design

RNAi-based anti-viral drugs employing siRNA molecules are designed and used to target viral disease of cattle. For each viral agent, a viral protein necessary for replication and/or contributing to disease pathogenesis is targeted. The gene sequences to be targeted for siRNA silencing are determined from known published viral genome and/or through gene sequencing. A panel of siRNA molecules are rationally designed based on the following general guidelines:

General Guidelines of Design

siRNA targeted sequences are usually 19-27 nt in length.

Regions to be targeted within 50-100 bp of the start codon and the termination codon are to be avoided.

Intron regions are avoided for targeting.

Stretches of 4 or more bases such as AAAA, CCCC are avoided.

Regions with GC content <about 30% or >about 60% are avoided.

Nucleotide repeats and low complex sequence are avoided.

Single nucleotide polymorphism (SNP) sites are avoided.

BLAST homology search will be used to avoid off-target effects on other genes or sequences and gene microarrays are used to assess off-target effects

The negative controls are designed by scrambling targeted siRNA sequence. The control RNA are the same length and nucleotide composition as the siRNA but have at least 4-5 bases mismatched to the siRNA. The negative control after scrambling is BLAST homology searched to ensure the scrambling does not create new homology to other genes.

In creating siRNAs the first 23-nt sequence motif AA(N₁₉) are searched, and if no suitable sequence is found, then, the search is focused on an about 23-nt sequence motifNA(N₂₁) that is convert the 3′ end of the sense siRNA to TT

As an alternative strategy, siRNAs are made against searches for NAR(N₁₇)YNN

The target sequence are searched for GC content of 50%

(A=Adenine; T=Thymine; R=Adenine or Guanine (Purines); Y=Thymine or Cytosine (Pyrimidines); N=Any).

Overview of Rational siRNA Design:

By experimentally analyzing the silencing efficiency of siRNAs targeting the mRNA of two genes and correlating it with various sequence features of individual siRNAs, eight characteristics associated with siRNA functionality are identified. These characteristics are used by rational siRNA design to evaluate potential targeted sequences. Sequences with higher scores will have higher chance of success in RNAi. The table below lists the 8 criteria and the methods of score assignment.

TABLE 2
Eight criteria and methods of score assignment.

| | | Score | |
|---|---|---|---|
| Criteria | Description | Yes | No |
| 1 | Moderate to low (30%-52%) GC Content | 1 point | |
| 2 | At least 3 A/Us at positions 15-19 (sense) | 1 point/ | |
| | | per A or U | |
| 3 | Lack of internal repeats (Tm* <20iãC) | 1 point | |
| 4 | A at position 19 (sense) | 1 point | |
| 5 | A at position 3 (sense) | 1 point | |
| 6 | U at position 10 (sense) | 1 point | |
| 7 | No G/C at position 19 (sense) | | −1 point |
| 8 | No G at position 13 (sense) | | −1 point |

A sum score of 6 defines the cutoff for selecting siRNAs. All siRNAs scoring higher than 6 are acceptable candidates.
*Tm = 79.8 + 18.5 * log₁₀([Na⁺]) + (58.4 * GC %/100) + (11.8 * (GC %/100)²) − (820/Length)
For example, the Tm can be calculated as follows for the siRNA UUCUCCAGCUUCUAAAAUA Tm = 79.8 + 18.5 * log₁₀(0.05) + (58.4 * 31.6/100) + (11.8 * (31.6/100)²) − (820/19) Tm = 32.19

Targeted inhibition of gene expression may be implemented via the use of RNA interference molecules, where the nucleotide sequence of such compounds are related to the nucleotide sequences of DNA and/or RNA of genes that are involved in the initiation, transcription, translation or replication of the viruses. In certain embodiments, an RNA interference (RNAi) molecule is used to decrease gene expression in BRSV. The methods described herein are generally applicable to any members of the Paramyxoviridae virus family.

Pharmaceutical compositions for use in accordance with the present invention may be formulated in conventional manner using one or more physiologically acceptable carriers or excipients. Thus, the RNA molecules of the present invention may be formulated for administration by inhalation or insufflation (either through the mouth or the nose) or oral, parenteral or mucosal (such as buccal, vaginal, rectal, sublingual) administration. Methods of preparing pharmaceutical formulations are well known.

An embodiment of the invention provides a pharmaceutical pack or kit comprising one or more containers filled with the RNA molecules of the present invention. Optionally associated with such container(s) can be a notice in the form prescribed by a governmental agency regulating the manufacture, use or sale of pharmaceuticals or biological products, which notice reflects approval by the agency of manufacture, use or sale for bovine administration. An embodiment of the invention provides kits that can be used in the above methods. In one embodiment, a kit comprises RNA molecules of the present invention, in one or more containers, and one or more other prophylactic or therapeutic agents useful for the treatment of BRSV, in one or more containers.

Embodiments of the invention also include vectors comprising polynucleotide molecules of the invention, as well as a host cell transformed with such vectors. Any of the polynucleotide molecules of the invention may be joined to a vector, which generally includes a selectable marker and an origin of replication, for propagation in a host. Vectors include DNA encoding any of the RNA described herein, operably linked to suitable transcriptional regulatory sequences, such as those derived from a mammalian, microbial, viral, or insect gene. Examples of regulatory sequences include transcriptional promoters, operators, or enhancers, mRNA ribosomal binding sites, and appropriate sequences which control transcription and translation. In yet another embodiment, an expression vector of the invention comprises a nucleic acid sequence encoding two or more siRNA sequences, which can be the same or different.

Dosage/Formulation/Administration

Pharmaceutical compositions for use in accordance with the present invention may be formulated in conventional manner using one or more physiologically acceptable carriers or excipients. Thus, the RNA molecules of the present invention may be formulated for administration by inhalation or insufflation (either through the mouth or the nose) (Thomas, M., J. J. Lu, J. Chen, and A. M. Klibanov. 2007. Non-viral siRNA delivery to the lung. Advanced Drug Delivery Reviews 59:124-133) or oral, parenteral or mucosal (such as buccal, vaginal, rectal, sublingual) or intravenous (Herweijer, H., and J. A. Wolff. 2006. Gene therapy progress and prospects: Hydrodynamic gene delivery. Gene Ther 14:99-107; Li, S. D., and L. Huang. Gene therapy progress and prospects: non-viral gene therapy by systemic delivery. Gene Ther 13:1313-1319) administration (Li, C. X., A. Parker, E. Menocal, S. Xiang, L. Borodyansky, and J. H. Fruehauf. 2006. Delivery of RNA interference. Cell Cycle 5:2103-2109; de Fougerolles, A., H.-P. Vomlocher, J. Maraganore, and J. Lieberman. 2007. Interfering with disease: a progress report on siRNA-based therapeutics. Nat Rev Drug Discov 6:443-453). Methods of preparing pharmaceutical formulations are well known (Li, C. X., A. Parker, E. Menocal, S. Xiang, L. Borodyansky, and J. H. Fruehauf. 2006. Delivery of RNA interference. Cell Cycle 5:2103-2109). Dosage of inhibitory nucleic acids may vary by route of administration, exemplary suitable dosages can range from about 0.5 mg/kg to about 5 mg/kg or greater.”

The inhibitory polynucleotide molecules may be naked (i.e. non-formulated) or formulated in a variety of carrier agents such as, but not limited to, polyethylenimine (PEI) and other polymers (Thomas, M., J. J. Lu, J. Chen, and A. M. Klibanov. 2007. Non-viral siRNA delivery to the lung. Advanced Drug Delivery Reviews 59:124-133; Howard, K. A., and J. Kjems. 2007. Polycation-based nanoparticle delivery for improved RNA interference therapeutics. Expert Opinion on Biological Therapy 7:1811-1822), nanoparticles, cationic lipids/liposomes (DOTAP, DOPE, cholesterol, etc.) (Howard, K. A., and J. Kjems. 2007. Polycation-based nanoparticle delivery for improved RNA interference therapeutics. Expert Opinion on Biological Therapy 7:1811-1822; Zhang, S., B. Zhao, H. Jiang, B. Wang, and B. Ma. 2007. Cationic lipids and polymers mediated vectors for delivery of siRNA. Journal of Controlled Release 123:1-10), peptide (Meade, B. R., and S. F. Dowdy. 2007. Exogenous siRNA delivery using peptide transduction domains/cell penetrating peptides. Advanced Drug Delivery Reviews 59:134-140; Moschos, S. A., A. E. Williams, and M. A. Lindsay. 2007. Cell-penetrating-peptide-mediated siRNA lung delivery. Biochemical Society Transactions 035:807-810) (e.g. Penetratin™ 1 (MPBiomedicals, Solon, Ohio)) protein/immunoglobulin (Liu, B. 2007. Exploring cell type-specific internalizing antibodies for targeted delivery of siRNA. Brief Funct Genomic Proteomic 6:112-119), or polyelectrolyte transfection reagents. Compositions may include conjugation of carriers (e.g. peptides or cholesterols) or formulation (mixing).

In certain embodiments, the RNAi polynucleotides may also be combined with other therapeutic agents or compounds (e.g. antibiotics) as co-administration or co-formulation components.

In certain other embodiments, the therapeutic efficacy of the RNAi compositions may be enhanced by non-specific anti-viral mechanisms, such as, for example, synergistic effects based on interferon gamma induction.

In certain embodiments, the pharmaceutical formulations comprise interfering RNAs, or salts thereof, of the invention up to 99% by weight mixed with a physiologically acceptable carrier medium such as water, buffer, saline, glycine, hyaluronic acid, mannitol, and the like.

Interfering RNA embodiments of the present invention can be administered as solutions, suspensions, or emulsions.

Generally, an effective amount of the interfering RNAs of embodiments of the invention results in an extracellular concentration at the surface of the target cell of from 100 pM to 1000 nM, or from 1 nM to 400 nM, or from 5 nM to about 100 nM, or about 10 nM. The dose required to achieve this local concentration will vary depending on a number of factors including the delivery method, the site of delivery, the number of cell layers between the delivery site and the target cell or tissue, whether delivery is local or systemic, etc. The concentration at the delivery site may be considerably higher than it is at the surface of the target cell or tissue.

Topical compositions are delivered to the surface of the target organ one to four times per day, or on an extended delivery schedule such as daily, weekly, bi-weekly, monthly, or longer, according to the routine discretion of a skilled clinician. The pH of the formulation is about pH 4-9, or pH 4.5 to pH 7.4.

An effective amount of a formulation may depend on factors such as the age, race, and sex of the subject, the severity of the viral infection, the rate of target gene transcript/protein turnover, the interfering RNA potency, and the interfering RNA stability, for example. In one embodiment, the interfering RNA is delivered topically to a target organ and reaches target protein-containing tissue at a therapeutic dose thereby ameliorating a viral-infection/replication process.

Acceptable carriers: An acceptable carrier refers to those carriers that cause at most, little to no ocular irritation, provide suitable preservation if needed, and deliver one or more interfering RNAs of the present invention in a homogenous dosage. An acceptable carrier for administration of interfering RNA of embodiments of the present invention include the cationic lipid-based transfection reagents TransIT®-TKO (Mirus Corporation, Madison, Wis.), LIPOFECTIN®, Lipofectamine, OLIGOFECTAMINE® (Invitrogen, Carlsbad, Calif.), or DHARMAFECT® (Dharmacon, Lafayette, Colo.); polycations such as polyethyleneimine; cationic peptides such as Tat, polyarginine, or Penetratin (Antp peptide); or liposomes. Liposomes are formed from standard vesicle-forming lipids and a sterol, such as cholesterol, and may include a targeting molecule such as a monoclonal antibody having binding affinity for endothelial cell surface antigens, for example. Further, the liposomes may be PEGylated liposomes.

The exemplary interfering RNAs may be delivered in solution, in suspension, or in bioerodible or non-bioerodible delivery devices. The interfering RNAs can be delivered alone or as components of defined, covalent conjugates. The interfering RNAs can also be complexed with cationic lipids, cationic peptides, or cationic polymers; complexed with proteins, fusion proteins, or protein domains with nucleic acid binding properties (e.g., protamine); or encapsulated in nanoparticles. Tissue—or cell-specific delivery can be accomplished by the inclusion of an appropriate targeting moiety such as an antibody or antibody fragment.

For ophthalmic, otic, or pulmonary delivery, an exemplary interfering RNA may be combined with ophthalmologically, optically, or pulmonary acceptable preservatives, co-solvents, surfactants, viscosity enhancers, penetration enhancers, buffers, sodium chloride, or water to form an aqueous, sterile suspension or solution. Solution formulations may be prepared by dissolving the interfering RNA in a physiologically acceptable isotonic aqueous buffer. Further, the solutions may include an acceptable surfactant to assist in dissolving the inhibitor. Viscosity building agents, such as hydroxymethyl cellulose, hydroxyethyl cellulose, methylcellulose, polyvinylpyrrolidone, or the like may be added to the compositions of the present invention to improve the retention of the compound.

In certain embodiments, preparation of a sterile ointment formulation can include the combination of the exemplary interfering RNA with a preservative in an appropriate vehicle, such as mineral oil, liquid lanolin, or white petrolatum.

Sterile gel formulations may be prepared by suspending the interfering RNA in a hydrophilic base prepared from the combination of, for example, CARBOPOL®-940 (BF Goodrich, Charlotte, N.C.), or the like, according to methods known in the art. VISCOAT® (Alcon Laboratories, Inc., Fort Worth, Tex.) may be used for intraocular injection, for example.

Other compositions of the present invention may contain penetration enhancing agents such as cremephor and TWEEN® 80 (polyoxyethylene sorbitan monolaureate, Sigma Aldrich, St. Louis, Mo.), in the event the interfering RNA is less penetrating in the organ or tissue of interest.

An embodiment of the invention also includes an expression vector comprising a polynucleotide encoding siRNA sequence of the invention in a manner that allows expression of the polynucleotide (Li, C. X., A. Parker, E. Menocal, S. Xiang, L. Borodyansky, and J. H. Fruehauf 2006. Delivery of RNA interference. Cell Cycle 5:2103-2109; de Fougerolles, A., H.-P. Vornlocher, J. Maraganore, and J. Lieberman. 2007. Interfering with disease: a progress report on siRNA-based therapeutics. Nat Rev Drug Discov 6:443-453; Amarzguioui, M., J. J. Rossi, and D. Kim. 2005. Approaches for chemically synthesized siRNA and vector-mediated RNAi. FEBS Letters 579:5974-5981). An embodiment ofthe invention also includes a host cell, for example a human cell, including an expression vector contemplated by the invention. In yet another embodiment, an expression vector of the invention comprises a nucleic acid sequence encoding two or more siRNA sequences, which can be the same or different.

Routes of Administration

A variety of protocols are available for in vivo delivery and administration of the exemplary RNAi polynucleotides. Inhibitory nucleic acids have been applied in vivo in animal models for a variety of infectious diseases including influenza virus (Tompkins, S. M., C. Y. Lo, T. M. Tumpey, and S. L. Epstein. 2004. Protection against lethal influenza virus challenge by RNA interference in vivo. Proc Natl Acad Sci U S A 101:8682-8686) and respiratory virus (Bitko, V., A. Musiyenko, O. Shulyayeva, and S. Barik. 2005. Inhibition of respiratory viruses by nasally administered siRNA. Nat Med 11:50-55). Moreover, inhibitory nucleic acids are in human clinical trial for a variety of diseases, including RSV, HIV, and Acute Macular Degeneration (AMD) (de Fougerolles, A., H.-P. Vomlocher, J. Maraganore, and J. Lieberman. 2007. Interfering with disease: a progress report on siRNA-based therapeutics. Nat Rev Drug Discov 6:443-453; Rossi, J. J., C. H. June, and D. B. Kohn. 2007. Genetic therapies against HIV. Nat Biotech 25:1444-1454).

In certain embodiments, interfering RNA may be delivered, for example, via aerosol, buccal, dermal, intradermal, inhaling, intramuscular, intranasal, intraocular, intrapulmonary, intravenous, intraperitoneal, nasal, ocular, oral, otic, parenteral, patch, subcutaneous, sublingual, topical, or transdermal administration. In certain other embodiments, administration may be directly to the lungs, via, for example, an aerosolized preparation, and by inhalation via an inhaler or a nebulizer, for example.

The compositions can be delivery prophylactically (i.e. prior to exposure or infection to reduce the likelihood or severity of infection) or therapeutically (after infection). Composition treatment can be a stand alone monotherapy or be given in combination with other anti-viral therapies known and practiced in the art.

In certain further embodiments, modes of administration can include tablets, pills, and capsules, all of which are capable of formulation by one of ordinary skill in the art.

Kits:

Aspects and embodiments of the present disclosure also provide a kit that includes reagents for attenuating the expression of an mRNA in a subject. The kit can contain an siRNA, miRNA or an shRNA expression vector. For siRNAs and non-viral shRNA expression vectors the kit also may contain a transfection reagent or other suitable delivery vehicle. For viral shRNA expression vectors, the kit may contain the viral vector and/or the necessary components for viral vector production (e.g., a packaging cell line as well as a vector comprising the viral vector template and additional helper vectors for packaging). The kit may also contain positive and negative control siRNAs or shRNA expression vectors (e.g., a non-targeting control siRNA or an siRNA that targets an unrelated mRNA). The kit also may contain reagents for assessing knockdown of the intended target gene (e.g., primers and probes for quantitative PCR to detect the target mRNA and/or antibodies against the corresponding protein for western blots). Alternatively, the kit may comprise an siRNA sequence or an shRNA sequence and the instructions and materials necessary to generate the siRNA by in vitro transcription or to construct an shRNA expression vector.

In certain embodiments, the instant disclosure provides a pharmaceutical pack or kit comprising one or more containers filled with the RNA molecules of the present invention. Optionally associated with such container(s) can be a notice in the form prescribed by a governmental agency regulating the manufacture, use or sale of pharmaceuticals or biological products, which notice reflects approval by the agency of manufacture, use or sale for veterinary administration. An embodiment of the invention provides kits that can be used in the above methods. In one embodiment, a kit comprises RNA molecules of the present invention, in one or more containers, and one or more other prophylactic or therapeutic agents useful for the treatment of Paramyxovirus in one or more containers.

In certain other embodiments, a pharmaceutical combination for co-administration or co-formulation is provided that includes, for example, a packaged combination comprising: an interfering RNA composition, a therapeutic agent, and an acceptable carrier. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### EXAMPLES

### Example 1

### Testing of Rational-Designed siRNA

siRNA molecules which are designed as indicated above are tested individually and as a pool (n=2-4 siRNAs) of siRNAs to evaluate their efficacy to decrease viral replication in appropriate cell lines. Commercially available siRNA transfection reagent and protocols are used as guidelines. Various siRNA concentrations are transfected into cell lines prior (6-12 h) to virus infection. Successful silencing are determined by decreased or ablated mRNA expression, and/or concomitant protein expression, and/or decrease in virus replication determined in vitro. Various concentrations of siRNAs are administered by intranasal, intravenous, ocular, and/or oral instillation. This process can be dependent on the virus type and should be generally related to the natural route of infection. After administration of siRNA, cattle are challenged with the virus from which the respective siRNA drug candidates are based using routes proven to cause identifiable clinical disease in pilot studies. Control cattle are administered a mismatched and/or irrelevant siRNA and similarly challenged. Clinical scores are determined at various time-points post-challenge as appropriate to provide a measurement for drug efficacy. An endpoint for drug efficacy are confirmation of decreased virus replication and/or virus shedding. Tissue of the relevant organ systems are evaluated at necropsy to confirm efficacy of siRNA treatment to decrease pathology due to viral infection. In subsequent studies, RNAi-based drugs are evaluated for their potential to treat active virus infection. In these studies, cattle are treated with various doses of siRNA at various time points following infection by intranasal, intravenous, ocular and/or by oral instillation to determine siRNA efficacy to reduce disease and or virus load.

Proof-of-concept is demonstrated against bovine respiratory syncytial virus (BRSV); in particular, the siRNAs target the viral phosphoprotein (P), which is a component of the viral polymerase and is required for the virus replication. In vitro screening of siRNA drug candidates is performed using Madin-Darby bovine kidney (MDBK) cells that are transfected with various concentrations of the siRNA drug candidates using commercial transfection reagents. Both individual and pooled siRNA against the BRSV P gene are tested for efficacy and the cells are infected with isolates of BRSV. One of these isolates has been confirmed to cause disease similar to that in naturally infected cattle. The cytopathic effect consistent with BRSV infection is quantified in treated and untreated MDBK cells, and siRNA efficacy is measured by decreased cytopathic effect and by a reduction in virus plaque formation determined by a virus plaque assay. Lead siRNA drug candidates that effectively silence BRSV in vitro are administered at various concentrations to cattle by intranasal instillation; control cattle are similarly treated with mismatched or irrelevant siRNA. Subsequently, siRNA-treated cattle are challenged with BRSV by aerosol exposure, and clinical signs and viral shedding is evaluated for 10 days following infection. Pathologic changes in the lung are evaluated in both groups by postmortem at day 10 following infection.

### Example 2

### Assessment of Viral Shedding Data

Confirmation of nasal shedding by cattle of bovine respiratory syncytial virus (BRSV) has been problematic using traditional methods. In the present invention, various methods of BRSV identification from experimentally infected calves were compared. Virus isolation (VI) followed by immunohistochemistry (IHC), direct immunofluorescent antibody staining (IFA), or real time reverse transcriptase-polymerase chain reaction (RT-PCR) of nasal swabs were compared for frequency of virus identification and determination of relative amounts of virus shed. Eight calves were challenged with a clinical isolate of BRSV, then sampled and scored for clinical signs of disease for 7 days. Three sterile cotton swabs were inserted into alternate nostrils daily from day 0 (prior to infection) through day 7. Nasal swabs were immediately placed into 1 ml of tissue culture media, vigorously mixed, and transported on ice to the lab. Triplicate 100 μl aliquots of media from one swab were placed on bovine turbinate cells in 48-well plates; 3 serial ten-fold dilutions were made, the plate was incubated 10 days prior to IHC, and titers were calculated. The other two samples were centrifuged to pellet cells. RNA was extracted from the cell pellet from one sample for RT-PCR for BRSV N protein mRNA; results were expressed as N-fold change of AACt value. The pellet from the third sample was applied to a glass slide for direct IFA with semi-quantitative scoring. On day 7, calves were euthanized and lung tissue was collected for virus identification by IHC and RT-PCR. Virus was identified in nasal swabs from at least one calf by all three methods; however, IFA and RT-PCR detected virus in at least 7 of 8 calves, while VI only identified virus in 1 calf. Relatively more virus was identified by IFA or RT-PCR than by VI. Relative amount of nasal virus shedding correlated well with clinical signs, gross pathology, and lung virus identification by RT-PCR. BRSV identification and quantification by nasal swab IFA or RT-PCR were sensitive and reliable ways to identify virus shedding by infected calves.

This viral shedding data demonstrates that the results of the viral challenges can be measured from cattle in vivo. The clinical signs and lung pathology seen in the Group 1 calves were typical of that seen in naturally-occurring BRSV infection. This indicates that this method is a good “life-like” model of infection with which to show the efficacy of small interfering RNA (siRNA) treatment.

### Example 3

### in vitro Inhibition of BRSV Replication

siRNA directed to the P gene protein of BRSV reduces BRSV replication as determined by a reduction in viral plaque formation.

Materials and Methods

Virus and Cell Culture. Madin-Darby Bovine Kidney (MDBK) cells obtained from Merck-Merial were grown in Dulbecco's Modified Essential Medium (DMEM) (Cellgro) with 5% fetal calf serum. MDBK cells were infected with strain BRSV A51908 obtained from American Type Culture Collection (Manassas, Va.) at a multiplicity of infection (MOI) of approximately 1.0 at 70-80% cell confluency. For the infection step, the media were removed from the MDBK cells, followed by washing the MDBK cells in Hanks Balanced Salt Solution (HBSS), and then added the BRSV directly to the MDBK cells to adsorb for 2 hours at 37° C. in 5% CO₂. After the 2 hour incubation, complete medium was added. Tissue culture flasks of infected MDBK cells were grown at 37° C. in 5% CO₂ and harvested when approximately 50% of the cell monolayer demonstrated cytopathic effect (CPE). Viral stocks were obtained by freeze/thawing the infected cells three times on ice and centrifuging to clarify the virus from the cell debris. Stocks are then aliquoted and stored in liquid nitrogen storage until use.

BRSV siRNA-P transfection in 24-well plates using TransIT-TKO. The P gene sequence of BRSV was submitted to Ambion (The Woodlands, Tex.) and three siRNAs to a conserved region of the P gene were generated based on a proprietary algorithm. Sequences used were as follows: siRNA1, sense 5′-GCAACCAAGUUUCUUGAAUtt-3′, antisense 3′-ttCGUUGGUUC AAAGAACUUA-5′; siRNA2, sense 5′-CCAACCAAGUGAGAUCAAUtt-3′, antisense 3′-ttGGUUGGUUCACUCUAGUUA-5′; and siRNA3, sense 5′-GCCUUUGGUAAGCUUCA AAtt-3′, antisense 3′-ttCGGAAACCAUUCGAAGUUU-5′. As a negative control an siRNA specific to influenza was included: nucleoprotein NP-1496 (siNP), sense 5′-GGAUCUUAUUU CUUCGGAGdTdT-3′, antisense 5′-dTdTCCUAGAAUAAAGAAGCCUC-3′. Twenty-four hours prior to the assay, 24-well flat-bottom plates of MDBK cells were seeded to obtain 80-90% confluency. The cells were then transfected with appropriate siRNAs. Briefly, in serum-free medium, TransIT-TKO® transfection reagent (Mirus Bio Corp., Madison, Wis.) was allowed to complex with the respective siRNAs to yield a final concentration of 100 nM. Cells were incubated in an incubator for 6 hours at 37° C. in 5% CO₂. At 6 hours post-transfection, the siRNA mixtures were removed and washed with HBSS. BRSV was prepared in serum free DMEM and each well was infected with 100 pfu/well in 100 μl media. At 2 hours post-infection, the virus was removed, cells were overlayed with 2% complete methylcellulose, and incubated for 6 days at 37° C. in 5% CO₂. After 6 days, virus plaques were enumerated using a standard immunostaining plaque assay (described below). Each siRNA was tested in quadruplicate and reduction in total plaque numbers was normalized to cells treated with TransIT-TKO® and BRSV.

Immunostaining Plaque Assay for Bovine Respiratory Syncytial Virus. Six hours prior to virus infection, 24-well plates of MDBK cells were seeded to obtain 80-90% confluency and were transfected with individual siRNAs using TransIT-TKO® transfection reagent per the manufacturer's suggested protocol. Subsequently, cells were washed one time with HBSS and inoculated with 200 μl/well of virus in serum free DMEM. Virus was allowed to absorb for 2 hours at 37° C. After 2 hours, the cells were overlayed with 1 ml/well of complete 2% methylcellulose media and incubated for 6 days at 37° C. in 5% CO₂. On day 6 post-infection, the overlay was removed and the cells were fixed with a cold acetone:methanol solution (60:40). A monoclonal antibody reactive to the F glycoprotein of BRSV (MAb 8G12; provided by Clayton Kelling, Department of Veterinary & Biomedical Sciences, University of Nebraska-Lincoln) was used for detection of BRSV plaque formation. Briefly, a 1:200 dilution of MAb 8G12 was added to wells and allowed to react for 2 hours at 37° C. After incubation the cells were washed three times with phosphate buffered saline (PBS) containing 5% Tween -20 (Sigma-Aldrich, St. Louis, Mo.). A secondary goat anti-mouse IgG (H+L) alkaline phosphatase conjugated antibody was added and allowed to react for 1-2 hours at 37° C. (Pierce Biotechnology Inc., Rockford, Ill.). Cells were washed again and treated with Vector® Black alkaline phosphatase substrate (Vector Laboratories, Burlingame, Calif.) for 20 minutes until color change was present. Plaques were then counted manually using an inverted light microscope at 4×magnification.

Results

The data indicate that a six hour treatment with siRNAs directed toward the P gene of BRSV was able to reduce virus replication as indicated by a significant reduction in viral plaque formation at a concentration of 100 nM. All assays were run in quadruplicate, and percentage reduction was normalized to the plaque formation in cells treated with TransIT-TKO® transfection reagent and infected with BRSV.

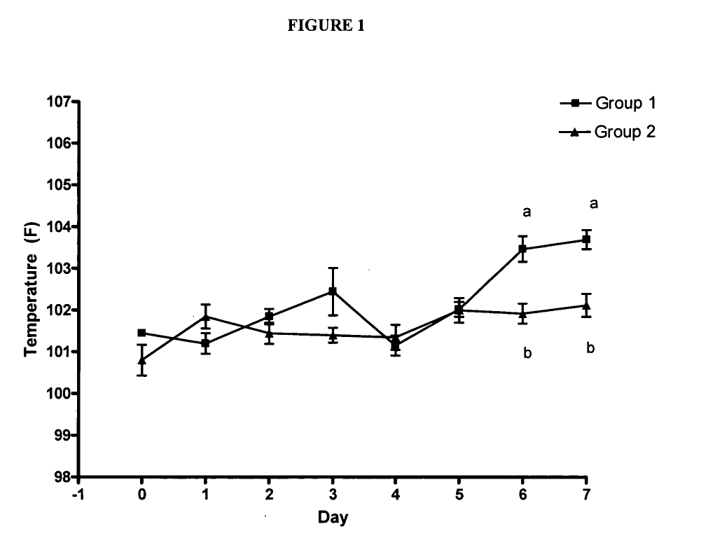

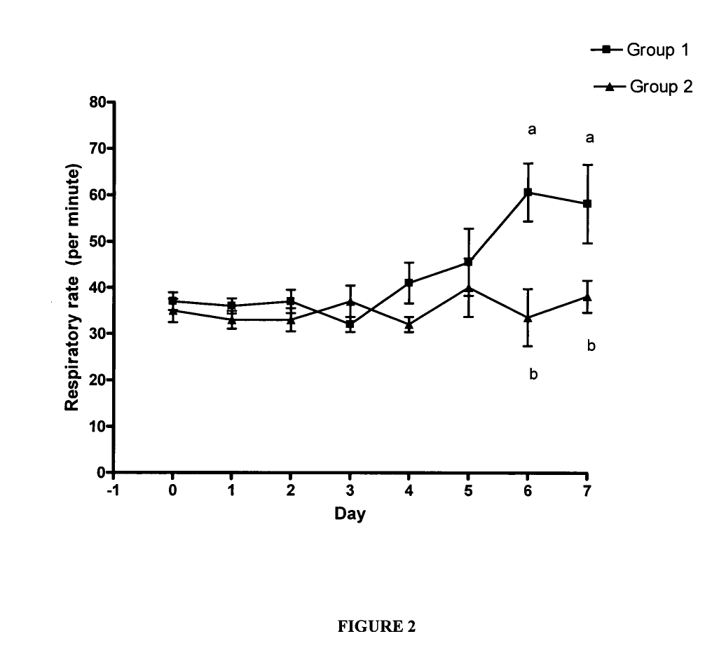

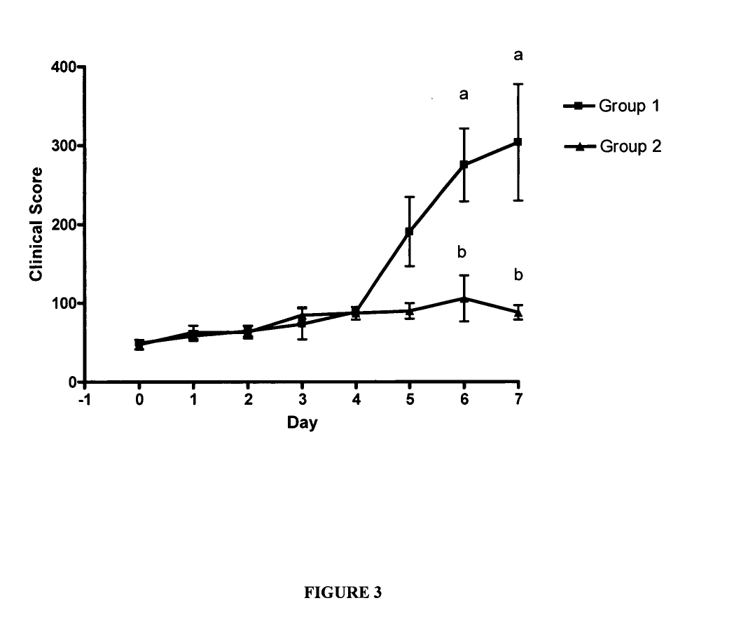

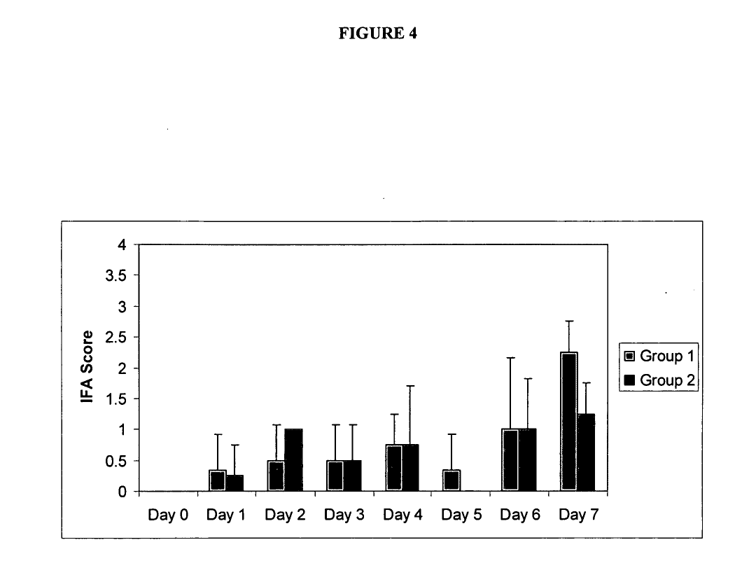

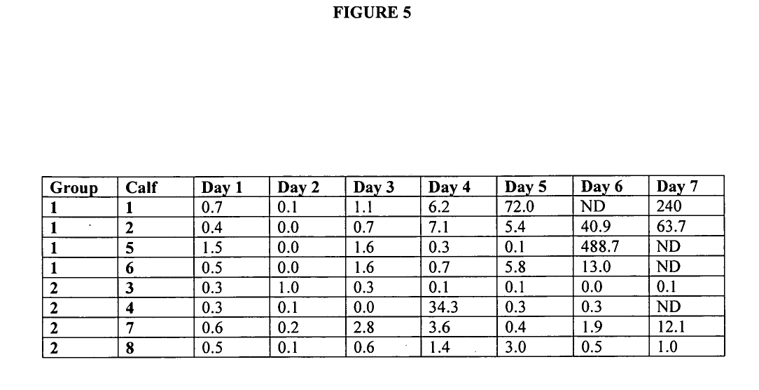

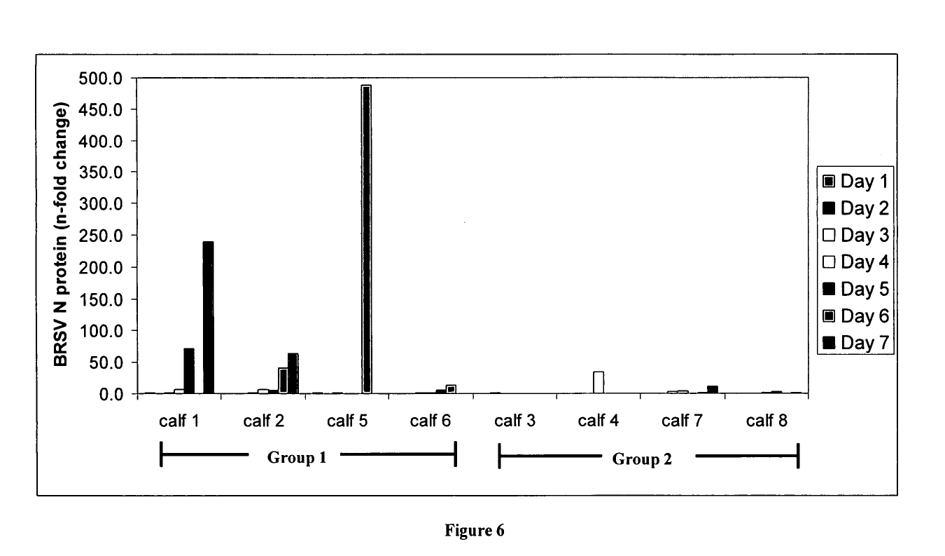

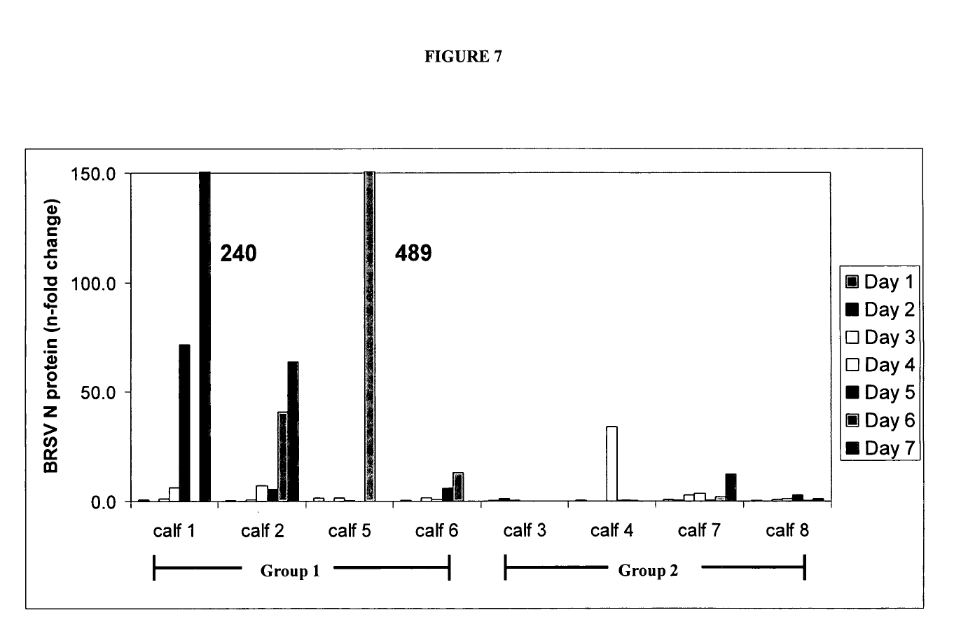

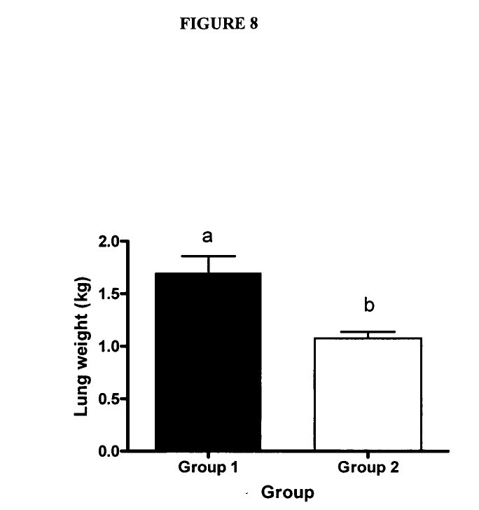

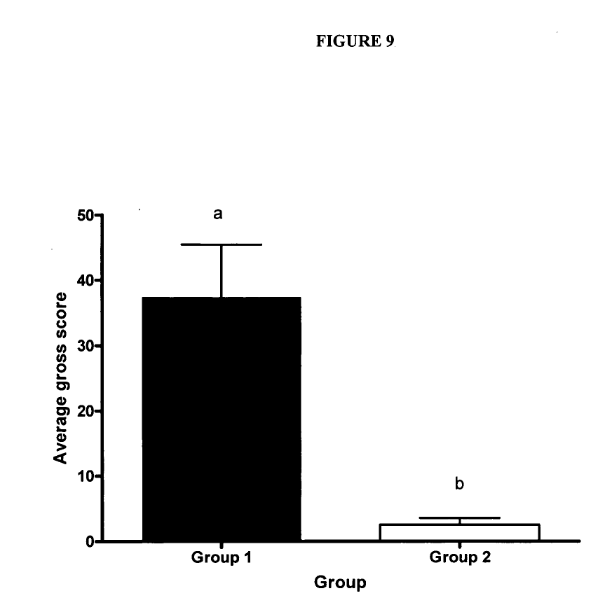

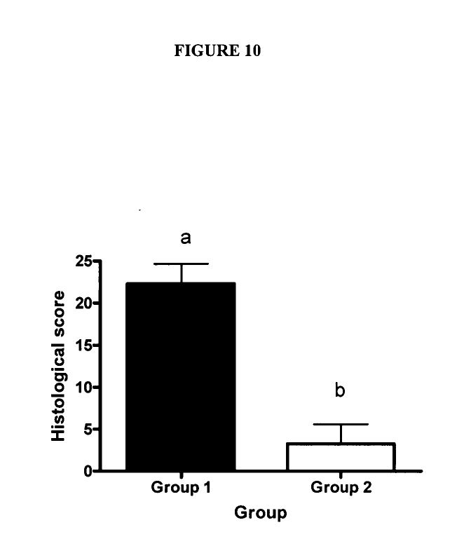

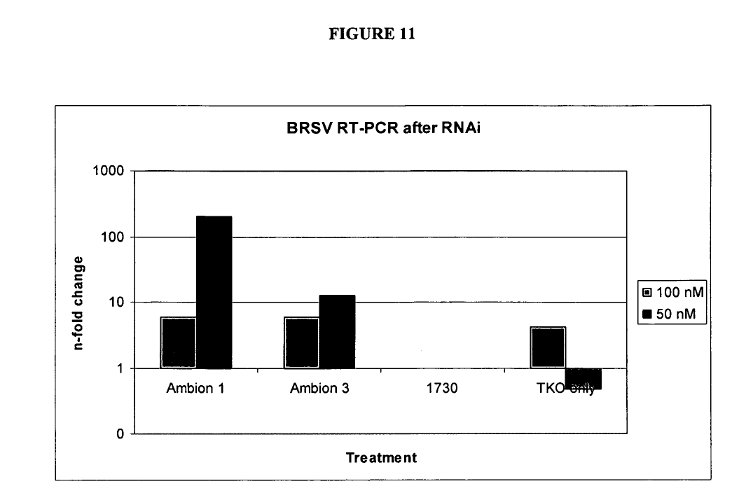

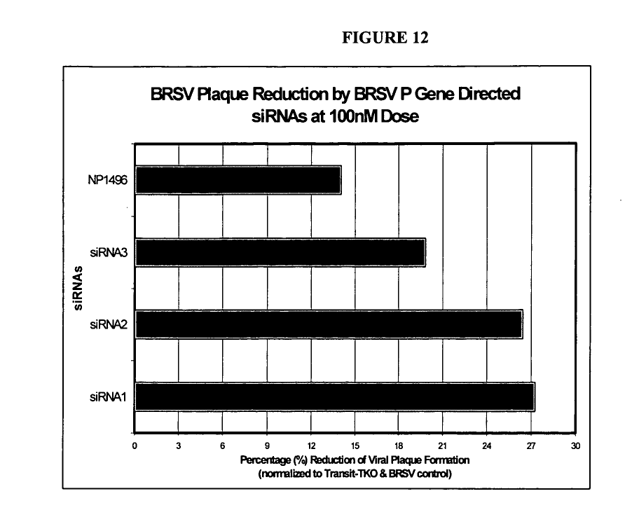

Of the three siRNAs screened, the reduction in plaque formation was observed in the range of 19-27%. Administration of two of the siRNAs, siRNA 1 and siRNA2, demonstrated plaque reduction greater than 25%. Taking into consideration the Type I anti-viral interferon response that could be caused by treatment of the MDBK cells with siRNAs (Reynolds et al., RNA 2006, 12(6):988-93), the NP-1496 siRNA directed against influenza (negative control) also demonstrated plaque reduction of approximately 13%. Subtracting the non-virus specific response still yields reduction in plaque formation in the range of 6-14% for siRNA1 and siRNA2 (FIG. 1).

siRNAs directed against the P gene of BRSV were effective at reducing BRSV (ATCC strain A51908) replication in MDBK cells as determined by a reduction in viral plaque formation. These results indicate that siRNAs against BRSV are effective anti-viral drugs, and that increasing the siRNA treatment dose or pooling the siRNAs may be a mechanism to enhance siRNA efficacy.

### Example 4

### in vivo Inhibition of BRSV Replication

Calves are treated intranasally with 1) low dose siRNA-P (0.05 mg/kg, 3 mg); 2) high dose siRNA-P (0.5 mg/kg, 30 mg); 3) missense siRNA-P (30 mg); or 4) saline (n=4 in each group) 4 hours prior to BRSV challenge. A second group of calves are treated intranasally with 1) low dose siRNA-P (0.05 mg/kg, 3 mg); 2) high dose siRNA-P (0.5 mg/kg, 30 mg); 3) missense siRNA-P (30 mg); or 4) saline (n=4 in each group) subsequent to BRSV challenge.

Clinical signs of both groups are evaluated prior to siRNA treatment, prior to challenge, and daily for 7 days post challenge. Nasal swabs are collected daily for quantification of nasal virus shedding. Lung tissue are collected postmortem to determine BRSV titer by immunostaining plaque assay. RT-PCR for BRSV P are performed to confirm message degradation.

Necropsy of all calves are performed on day 7 post-challenge. Gross and histopathologic changes are recorded, and localization of BRSV are characterized by immunohistochemistry. mRNA for IFN-α and IFN-γ in the lungs of the calves are evaluated by reverse-transcriptase PCR (RT-PCR) of total RNA extracted from lung tissue collected at necropsy on day 7 post challenge. IFN-γ levels in supernatants of lung homogenates are determined by ELISA. To determine the impact of siRNA-P treatment on Th1/Th2-type cytokine repertoire expression, mRNA for Th1 (IL-2 and IL-12, as well as IFN-γ) and Th2 (IL-4, IL-5, IL-10) cytokines, and TNFα, are measured by RT-PCR.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

While a particular embodiment of the invention has been shown and described, numerous variations and alternate embodiments will occur to those skilled in the art. Accordingly, it is intended that the invention be limited only in terms of the appended claims.

The invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes to the claims that come within the meaning and range of equivalency of the claims are to be embraced within their scope.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. An isolated nucleic acid molecule comprising a polynucleotide sequence of any one of the sequences of Table 1 and/or a complement thereof.

2. The isolated nucleic acid molecule of claim 1 wherein said nucleic acid molecule is double-stranded.

3. The isolated nucleic acid molecule of claim 2 wherein said nucleic acid molecule is RNA.

4. A vector comprising the isolated nucleic acid molecule of claim 1.

5. An isolated host cell containing the vector of claim 4.

6. A formulation comprising a therapeutically effective amount of the polynucleotide of claim 1 operably-linked to one or more control elements, and a pharmaceutically acceptable carrier.

7. A kit comprising a container containing the formulation of claim 6.

8. A method for inhibiting BRSV infection or replication in a subject comprising administering a therapeutically effective amount of the polynucleotide of any one of claims 1-3 or the formulation of claim 6 to a subject in need thereof.

9. A method of treating a subject with a BRSV infection comprising administering a therapeutically effective amount of one or more of a nucleic acid molecule according to any one of claims 1 to 8 alone or in combination with another agent or compound.

10. A polynucleotide molecule that inhibits the expression of a viral protein wherein the polynucleotide comprises a nucleotide sequence of any one of the sequences of Table 1 and/or a complement thereof.

11. The polynucleotide molecule of claim 10 wherein said molecule is an RNA molecule.

12. The polynucleotide according to claim 11 wherein said molecule is double-stranded.

13. The polynucleotide according to claim 10 wherein the polynucleotide comprises an siRNA nucleotide sequence of any one of the sequences of Table 1 and/or a complement thereof.

14. The polynucleotide according to claim 10 wherein the polynucleotide comprises an miRNA nucleotide sequence of any one of the sequences of Table 1 and/or a complement thereof.

15. The polynucleotide according to claim 10 wherein the polynucleotide comprises an shRNA nucleotide sequence of any one of the sequences of Table 1 and/or a complement thereof.

16. The polynucleotide according to claim 13 wherein each RNA strand has a length from 19-25 nucleotides.

17. The polynucleotide according to claim 16 wherein each RNA strand has a length from 19-25 nucleotides.

18. The polynucleotide according to claim 17 wherein said RNA molecule is capable of target-specific nucleic acid modifications and wherein at least one strand has a 3′-overhang of 1-nucleotides.

19. The polynucleotide according to claim 12 wherein said double-stranded RNA molecule consists of a single double stranded region and single stranded regions of 1 to 5 nucleotides at the 3′ ends of at least one of the strands of said double-stranded RNA molecule.

20. The polynucleotide according to claim 11, wherein the RNA strands are chemically synthesized.

21. The polynucleotide according to claim 11, wherein the RNA strands are enzymatically synthesized.

22. The polynucleotide according to claim 11, wherein the RNA strands are purified.

23. The polynucleotide according to claim 12, wherein both strands of said double-stranded RNA each have a 3′-overhang from 1-5 nucleotides.

24. The polynucleotide according to claim 12, wherein both strands of said double-stranded RNA each have a 3′-overhang from 1-3 nucleotides.

25. The polynucleotide according to claim 12, wherein both strands of said double-stranded RNA each have a 3′-overhang of 2 nucleotides.

26. The polynucleotide according to claim 12, wherein both strands of said double-stranded RNA each have a 3′-overhang of 1 nucleotide.

27. The polynucleotide according to claim 12, wherein each strand has a length from 19-23 nucleotides.

28. The polynucleotide according to claim 12, wherein each strand has a length from 21-23 nucleotides.

29. The polynucleotide according to claim 12, wherein each strand has a length from 19-21 nucleotides.

30. The polynucleotide according to claim 12, wherein the double-stranded RNA comprises at least one sugar-modified ribonucleotide, wherein the 2′-OH group of said sugar-modified ribonucleotide is replaced by a group selected from H, OR, R, halo, SH, SR, NH2, NHR, N(R)2 or CN, wherein R is C1-C6 alkyl, alkenyl or alkynyl and halo is F, Cl, Br or I.

31. The polynucleotide according to claim 12, wherein the double stranded RNA comprises at least one backbone-modified ribonucleotide containing a phosphorothioate group.

32. A vector comprising the polynucleotide molecule of any one of claims 1 to 31.

33. An isolated host cell containing the vector of claim 32.

34. A formulation comprising a therapeutically effective amount of the polynucleotide of any one of claims 1-31 operably-linked to one or more control elements, and a pharmaceutically acceptable carrier.

35. A kit comprising a container containing the formulation of claim 34.

36. A method for inhibiting BRSV infection or replication comprising administering a therapeutically effective amount of the polynucleotide of any one of claims 1 to 31 or the formulation of claim 34 to a subject in need thereof.

37. A pharmaceutical composition comprising a therapeutic effective amount of any one of claims 1 to 31 and a pharmaceutically acceptable carrier.

38. A method of treating a subject with a BRSV infection comprising administering a therapeutically effective amount of one or more polynucleotide molecule according to any one of claims 1 to 31 alone or in combination with another agent or compound, and a pharmaceutically acceptable carrier.

39. The method of claim 36, wherein the composition is administered via an aerosol, buccal, dermal, intradermal, inhaling, intramuscular, intranasal, intraocular, intrapulmonary, intravenous, intraperitoneal, nasal, ocular, oral, otic, parenteral, patch, subcutaneous, sublingual, topical, or transdermal route.

40. A method of attenuating BRSV gene expression in a subject comprising: administering to the subject a composition comprising an effective amount of antiviral agent having a length of 19 to 27 nucleotides and a pharmaceutically acceptable carrier, the antiviral agent further comprising a region of at least 13, 14, 15, 16, 17, or 18 contiguous nucleotides having at least 80% sequence complementarity to, or at least 90% sequence identity with, any one of the sequences in Table 1 wherein the expression of the viral mRNA is attenuated thereby.

41. The method of claim 40, wherein the interfering RNA is one of an shRNA, an miRNA, or an siRNA.

42. The method of claim 40, wherein the interfering RNA is administered via in vivo expression from an expression vector capable of expressing the interfering RNA.

43. A method for preventing BRSV infection in BRSV-uninfected cattle comprising administering a therapeutically effective amount of the polynucleotide of any one of claims 1 to 31 or the formulation of claim 34 to a subject in need thereof.

44. A method of treating a subject with a BRSV infection comprising administering a therapeutically effective amount of one or more polynucleotide molecule according to any one of claims 1 to 31 alone or in combination with another agent or compound, and a pharmaceutically acceptable carrier.
